(19) United States
(12) Patent Application Publication
(10) Pub. No.: US 2016/0311743 A1
(43) Pub. Date: Oct. 27, 2016

(54) EXTRACTION OF POLYCOSANOL FROM CANE VINASSE

(71) Applicants: ROHM AND HAAS COMPANY, Philadelphia, PA (US); DOW GLOBAL TECHNOLOGIES LLC, Midland, MI (US)

(72) Inventors: Peter E.M. AERTS, Hulst (NL); Renato RAMOS, Sao Paulo (BR); Stephen PEASE, Ambler, PA (US); Stanislas BAUDOUIN, La Rochelle (FR)

(21) Appl. No.: 15/104,675

(22) PCT Filed: Dec. 9, 2014
(86) PCT No.: PCT/US2014/069251

§ 371 Date: Jun. 15, 2016

Related U.S. Application Data
(60) Provisional application No. 61/917,509, filed on Dec. 18, 2013.

(51) Int. Cl. C07C 29/74; B01D 15/38; B01D 61/02; B01D 15/36

(52) U.S. Cl. CPC C07C 29/74; B01D 15/365; B01D 15/3804; B01D 61/027; B01D 2311/2626

(57) ABSTRACT

Provided is a process for extracting polycosanol from cane vinasse comprising
providing a cane vinasse concentrate, wherein the amount of organic compounds in said cane vinasse concentrate is 35 g/1 or more, and wherein said cane vinasse concentrate is effectively free of solid particles having diameter of 10 μm or higher,
performing an ion exclusion chromatography step c) on said cane vinasse concentrate to produce an extract (EC) and a raffinate (RC),
performing a concentration step y) on said raffinate (RC) to produce a permeate (PY) and a retentate (RY), and
performing a separation step d)ii) on said retentate (RY) to produce a retentate (RDII) that contains polycosanol.

An important process for the production of ethanol is fermentation of sucrose that is extracted from sugar cane. A byproduct of this process is cane vinasse, which is a dilute aqueous liquid that contains salts and organic compounds. Cane vinasse typically has dark color, bad smell, and acidic pH. Currently, the usual method of disposition of the cane vinasse is to treat it as waste or as fertilizer. Common waste disposal methods for cane vinasse involve placement in soil or in lagoons. There is a growing concern that cane vinasse that is used as fertilizer or that is disposed of by these methods will cause contamination of soil and/or groundwater. It would be desirable to find a method of extracting valuable compounds from the cane vinasse instead of simply treating it as waste and instead of using it as a fertilizer of dubious value.

US 2002/0169311 describes a process in which an artificial vinasse solution is separated using a weakly acid cation exchange resin. The first peak eluted in the method of US 2002/0169311 is mixture of sodium chloride, sucrose, and betaine, and the second peak contains mannitol. It would be desirable to provide improved separation by providing a method that employed ion exclusion chromatography. It would also be desirable to provide a method that involved improving the ion exclusion chromatography process by up-concentrating cane vinasse prior to the ion exclusion chromatography. It would also be desirable to provide a method that allowed the extraction of a variety of valuable compounds such as one or more of inositol and polycosanol.

The following is a statement of the invention.

An aspect of the present invention is a process for extracting polycosanol from cane vinasse comprising providing a cane vinasse concentrate, wherein the amount of organic compounds in said cane vinasse concentrate is 35 g/l or more, and wherein said cane vinasse concentrate is effectively free of solid particles having diameter of 10 μm or higher; performing an ion exclusion chromatography step c) on said cane vinasse concentrate to produce an extract (EC) and a raffinate (RC); performing a concentration step y) on said raffinate (RC) to produce a permeate (PY) and a retentate (RY), and performing a separation step d)ii) on said retentate (RY) to produce a retentate (RDII) that contains polycosanol.

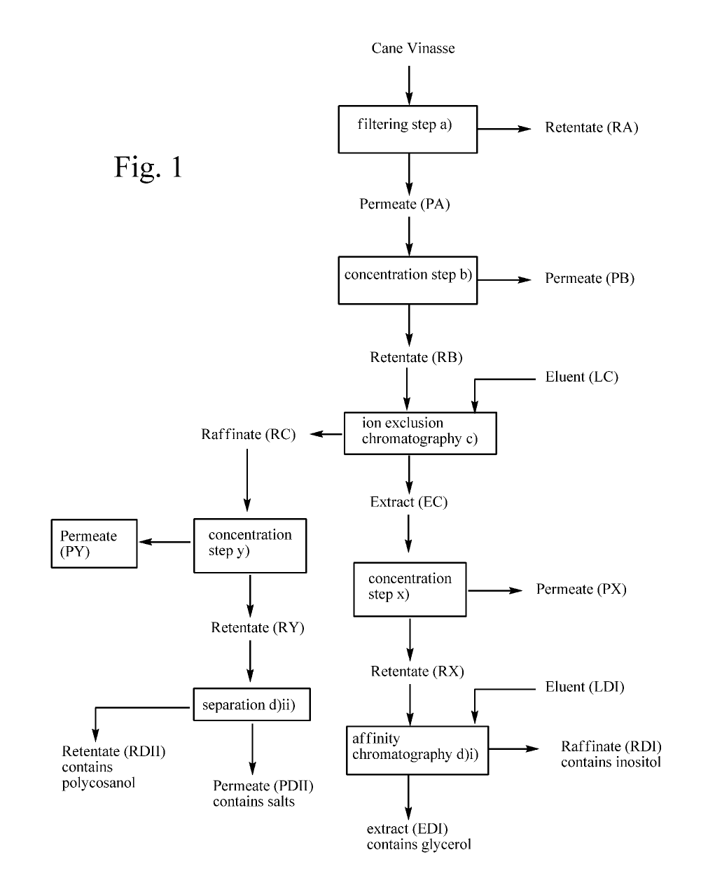

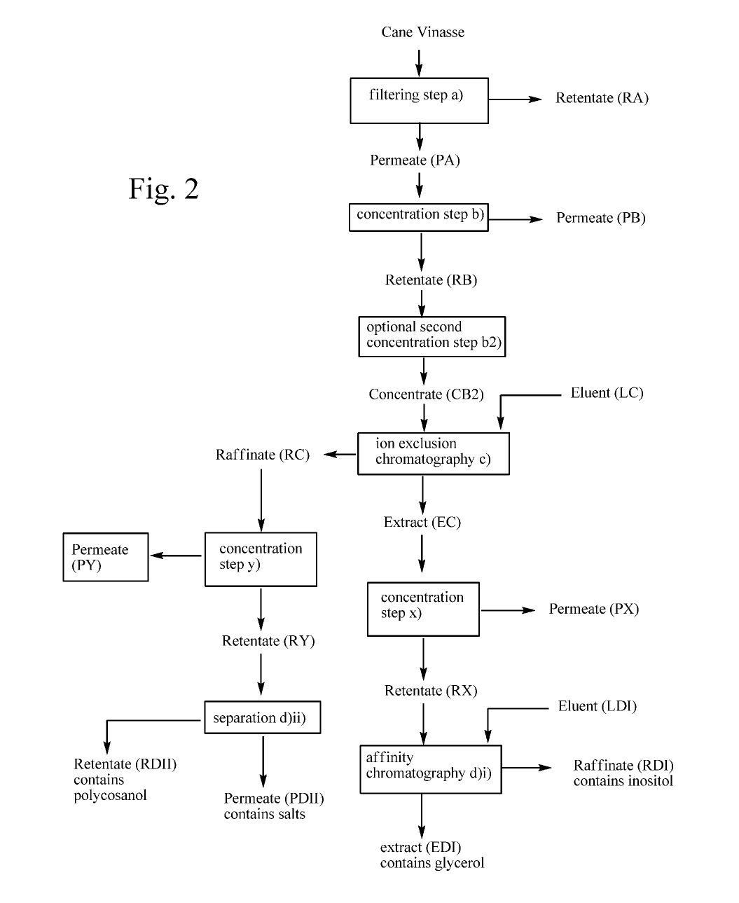

The following is a brief description of the drawings. FIG. 1 is a flow chart depicting the process of an embodiment of the present invention. FIG. 2 is a flow chart depicting an embodiment of the present invention that is the same as the embodiment depicted in FIG. 1 except that the embodiment depicted in FIG. 2 includes an optional concentration step b2) performed on retentate (RB), prior to ion exclusion chromatography step c).

The following is a detailed description of the invention.

An aqueous composition is a composition that has 50% or more water by weight based on the weight of the composition.

Cane vinasse is a byproduct of the process of extracting sucrose from sugar cane. Cane vinasse is an aqueous composition having 80% or more water by weight based on the weight of the cane vinasse. Preferably, cane vinasse has 90% or more water by weight based on the weight of the cane vinasse. Cane vinasse contains salts in the amount of 10 grams/liter (g/l) or more; preferably 20 g/l or more; more preferably 30 g/l or more. Cane vinasse preferably contains salts in the amount of 80 g/l or less; preferably 50 g/l or less. Cane vinasse contains organic compounds in the amount of 2 g/l or more; preferably 4 g/l or more; more preferably 8 g/l or more. Cane vinasse contains organic compounds in the amount of 30 g/l or less; preferably 20g/l or less. Among the organic compounds contained in cane vinasse, glycerol, inositol, and polycosanol are normally present.

As used herein, the term “polycosanol” refers to any compound having the structure CH₃—(CH2)ₙ—CH₂OH, where n is 24-34. The term polycosanol refers to any one of such compounds or to any mixture thereof. Polycosanol occurs naturally in cane vinasse.

Polycosanol contains octacosanol, triacontanol, or a mixture thereof.

In the process of the present invention, cane vinasse concentrate is used. The cane vinasse concentrate has organic compounds in the amount of 35 g/l or more; preferably 40 g/l or more; more preferably 50 g/l or more; more preferably 60 g/l or more; more preferably 70 g/l or more.

The cane vinasse concentrate is effectively free of solid particles having diameter of 10 μm or higher. If particles are not spherical, the diameter of the particle is the diameter of a sphere having the same volume as the particle. “Effectively free” of particles of a certain diameter or larger means having the amount of particles of that diameter or larger that would be present if the composition had been subjected to nanofiltration with a cut-off size of that diameter. Preferably, the cane vinasse concentrate is effectively free of solid particles having diameter of 5 μm or higher; more preferably effectively free of solid particles having diameter of 2 μm or higher.

A preferred method of producing cane vinasse concentrate is to use the steps a), b), and b2) as defined below.

In the process of the present invention, the cane vinasse is preferably subjected to filtering step a). The fluid that passes through the filter during filtering step a) is herein called the permeate (PA). The solid material retained on the filter medium is herein called the retentate (RA).

Preferably, filtering step a) is performed by microfiltration. Microfiltration is a process in which liquid is passed through the pores of a membrane; solid particles above a cut-off diameter are retained on the membrane. The cut-off diameter refers to the size at which 90% (generally) of the particles of that size are retained. The cut-off diameter may be assessed by measuring the pressure drop across a membrane and employing the Laplace equation; this method determines the size at which half the pores are larger while half the pores are smaller. Particles larger than the cut-off diameter are retained at an equal or higher percentage, and particles smaller than the cut-off diameter are retained at a smaller percentage. Preferably, the cut-off size is 10 μm or smaller; more preferably 5 μm or smaller; more preferably 2 μm or smaller; more preferably 1 μm or smaller. Preferably, the cut-off size is 0.01 μm or larger; more preferably 0.02 μm or larger; more preferably 0.05 μm or larger. Preferably, the membrane is ceramic.

In the preferred process of the present invention, the permeate (PA) is an aqueous composition that contains, among other things, one or more organic compounds. The permeate (PA) is subjected to concentrating step b). Concentrating step b) preferably removes water and possibly a relatively small amount of other materials from the permeate (PA) to form a water-rich component herein called permeate (PB). Preferably, permeate (PB) is either nearly pure water or a solution of one or more monovalent salts and a relatively small amount of other materials fully dissolved in water that, other than the dissolved monovalent salt(s), is nearly pure. Preferably, the amount of all materials other than water and dissolved monovalent salts in permeate (PB), by weight based on the weight of permeate (PB), is 20% or less; more preferably 5% or less; more preferably 1% or less, more preferably 0.5% or less. The concentrated material left behind when the water-rich component (permeate (PB)) is removed is herein called the retentate (RB).

Preferably, concentrating step b) is performed either by a process of reverse osmosis (RO) or by a process of nanofiltration (NF). RO and NF are processes in which pressure is used to drive pure or nearly pure water out of a sample of retentate (RB) by driving the water through a semipermeable membrane. In embodiments using reverse osmosis, the pure or nearly pure water that is driven through the semipermeable membrane is the permeate (PB), and the material left behind is the retentate (RB). The semipermeable membrane used in RO does not have permanent pores; the permeate diffuses through the semipermeable membrane material. RO is typically very effective at retaining nearly all solutes in the retentate, including monovalent ions. In NF, the semipermeable membrane may lack permanent pores or may have pores of 5 nm or less. In NF, the semipermeable membrane passes monovalent ions into the permeate more readily than does RO. NF is typically effective at retaining nearly all polyvalent ions and uncharged solutes in the retentate. NF generally operates at lower pressure than RO.

Optionally, retentate (RB) is subjected to a second concentration step b2). Second concentration step b2) produces concentrate (CB2). If second concentration step b2) is performed, concentrate (CB2) is subjected to ion exclusion chromatography c). Preferably, second concentration step b2), if it is performed, is a process of evaporation.

The retentate (RB) (or concentrate (CB2), if second concentration step b2) is performed) is subjected to a process of ion exclusion chromatography c). Ion exclusion chromatography c) separates mobile species into a raffinate (RC) fraction and an extract (EC) fraction. Ion exclusion chromatography involves elution using eluent (LC). The raffinate (RC) fraction is more highly mobile than the extract fraction (EC). Salts and relatively large organic compounds (those with 20 or more non-hydrogen atoms) will tend to pass through the chromatography medium relatively quickly. Therefore salts and some organic compounds including polycosanol will be found in the raffinate (RC). Smaller organic compounds will tend to be found in extract (EC). Glycerol and inositol will be found in extract (EC). The ion exclusion chromatography c) may be performed in discrete mode or in continuous mode. Continuous modes are preferred; more preferred is a simulated moving bed mode.

It is useful to characterize the composition that is subjected to ion exclusion chromatography c) (herein called composition PRE-C). Composition PRE-C will be retentate (RB) or concentrate (CB2) unless one or more optional step is performed on retentate (RB) or on concentrate (CB2) prior to performance of ion exclusion chromatography c). Preferably PRE-C is an aqueous composition. PRE-C preferably contains salts in the amount of 50 grams/liter (g/l) or more; more preferably 150 g/l or more; more preferably 250 g/l or more. PRE-C preferably contains salts in the amount of 400 g/l or less; more preferably 350 g/l or less. PRE-C preferably contains organic compounds in the amount of 25 g/l or more; more preferably 50 g/l or more; more preferably 75 g/l or more. PRE-C preferably contains organic compounds in the amount of 200 g/l or less; preferably 120 g/l or less.

Composition PRE-C is also known herein as the cane vinasse concentrate.

It is useful to compare the concentration of various compounds in cane vinasse to the concentration of the same compounds in PRE-C. For any specific compound or group of compounds, the quotient determined by dividing the concentration of that compound or group of compounds in PRE-C by the concentration of that compound or group of compounds in cane vinasse is known herein as the “Concentration Factor” for that compound or group of compounds. Preferably, the concentration factor of inositol is 5 or more; more preferably 6 or more. Preferably, the concentration factor of inositol is 12 or less; more preferably 10 or less. The preferred concentration factor for the total concentration of all dissolved salts is the same as the preferred concentration factor for inositol. The preferred concentration factor for the total concentration of all organic compounds is the same as the preferred concentration factor for inositol.

Preferably, ion exclusion chromatography c) is performed using a strong acid cation exchange (SAC) resin. Preferably, ion exclusion chromatography c) is performed using a cation exchange resin in the Na⁺ form or K⁺ form. Preferably, ion exclusion chromatography c) is performed using as the elution fluid (herein called eluent (LC)) either water or permeate (PB).

The present invention involves extraction of polycosanol by performing operations on raffinate (RC), preferably using steps y) and d)ii) as described below. Optionally, it may be desired to extract additional useful compounds, for example by use of steps x) and d)i) described below.

The step of concentration x) is preferably performed on extract (EC). Concentration x) produces permeate (PX) and retentate (RX). Retentate (RX) is then subjected to affinity chromatography d)i). Preferably, concentrating step x) is performed either by a process of reverse osmosis (RO) or by a process of nanofiltration (NF), as described herein above. In RO and NF, pressure is used to drive pure or nearly pure water out of a sample of retentate (RX) by driving the water through a semipermeable membrane. In embodiments using RO or NF, the pure or nearly pure water that is driven through the semipermeable membrane is the permeate (PX), and the material left behind is the retentate (PX). Preferred composition for permeate (PX) is the same as the preferred composition for permeate (PB).

The retentate (RX) is preferably subjected to a process of affinity chromatography d)i), which separates mobile species into a more-mobile raffinate (RDI) and a less-mobile extract (EDI). Affinity chromatograph d)i) involves the use of eluent (LDI). The raffinate (RDI) contains inositol, and the extract (EDI) contains glycerol. The affinity chromatography d)i) may be performed in discrete mode or in continuous mode. Continuous modes are preferred; more preferred is a simulated moving bed mode.

Preferably, affinity chromatography d)i) is performed using a strong acid cation exchange (SAC) resin. Preferably, affinity chromatography d)i) is performed using a cation exchange resin in the Ca⁺⁺ form. Preferably, affinity chromatography d)i) is performed using water as the elution fluid.

The extract (EDI) will contain solvent and, possibly, other compounds, in addition to glycerol. Preferably the solvent is water. It is contemplated that extract (EDI) will contain a usefully high concentration of glycerol and that the level of compounds other than solvent and glycerol will be low. The glycerol is preferably separated from such solvent and other compounds; this separation may be performed by familiar purification methods such as, for example, solvent evaporation.

The raffinate (RDI) will contain solvent and, possibly, other compounds, in addition to inositol. Preferably the solvent is water. It is contemplated that raffinate (RDI) will contain a usefully high concentration of inositol and that the level of compounds other than solvent and inositol will be low. The inositol is preferably separated from such solvent and other compounds; this separation may be performed by familiar purification methods such as, for example, solvent evaporation.

The raffinate (RC) (produced by the step of ion exclusion chromatography c)) is preferably subjected to concentration step y). Preferably, concentration step y) is a process of nanofiltration, reverse osmosis, evaporation, or a combination thereof. Concentration step y) produces a permeate (PY) and a retentate (RY). In the case of evaporation, water vapor is considered to be the permeate (PY).

Retentate (RY) is preferably subjected to a process of separation d)ii). The separation process produces a permeate (PDII) and a retentate (RDII). Permeate (PDII) contains salts, and retentate (RDII) also contains polycosanol. Preferred separation processes are nanofiltration, solvent extraction, and winterization; preferred is nanofiltration. In nanofiltration, the pore size is preferably 0.5 nm or larger. In nanofiltration, the pore size is preferably 2 nm or smaller. In nanofiltration, material that passes through the membrane is the permeate (PDII), and material that does not pass through the membrane is the retentate (RDII).

Prior to separation step d)ii), retentate (RY) is preferably mixed with one or more organic solvents. An organic solvent is a compound that is liquid at 25° C. and that has boiling point of 200° C. or lower. An organic solvent may be a pure compound or may be a mixture of compounds. Preferred are organic solvents in which polycosanol is soluble at 25° C. in an amount, per 100 grams of organic solvent, 1 gram or more; more preferably 5 grams or more. Preferred organic solvents are chloroform, ethanol, acetone, benzene, toluene, ethyl ether, petroleum ether, and mixtures thereof. More preferred is ethanol. Preferably, the amount of ethanol, per 100 grams of retentate (Y), is 60 grams or less; more preferably 40 grams or less. Preferably, the amount of ethanol, per 100 grams of retentate (Y), is 5 grams or more, more preferably 10 grams or more.

When separation step d)ii) is nanofiltration, it is preferably performed at pressure of 500 kPa (5 bar) or above; more preferably 1,000 kPa (10 bar) or above. When separation step d)ii) is nanofiltration, it is preferably performed at pressure of 8,000 kPa (80 bar) or below.

When separation step d)ii) is nanofiltration, it is preferably performed at temperature of 25° C. or above; more preferably 50° C. or above. When separation step d)ii) is nanofiltration, it is preferably performed at temperature of 80° C. or below.

It is contemplated that the solvent that is present in the composition that is subjected to separation step d)ii) is water or a mixture of water with one or more organic solvent. It is contemplated that some or all of this solvent will pass through the membrane and be found in the permeate (PDII). Some of the solvent may be retained and be found in the retentate (RDII). The retentate (RDII) may contain solvent and, possibly, other compounds, in addition to polycosanol. Preferably, the solvent, if present, contains water. It is contemplated that raffinate (RDII) will contain a usefully high concentration of polycosanol and that the level of compounds other than polycosanol will be low. The polycosanol is preferably separated from such solvent and other compounds; this separation may be performed by familiar purification methods, including but not limited to solvent exchange.

Also contemplated are embodiments in which one or more additional operations are performed in between any two of the above-described steps. Such an additional operation would be inserted between two of the above-described steps in manner analogous to the way in which the step of concentration x) may be inserted between ion exclusion chromatograph c) and affinity chromatograph d)i). Such additional steps may be, for example, one or more of concentration, purification, or a combination thereof.

The following are examples of the present invention.

### EXAMPLE 1

### Microfiltration of Cane Vinasse (Step a))

The feed was cane vinasse. Microfiltration was performed with KERASEP™ ceramic membranes from Novasep Process. A MicroKerasep™ pilot plant was used, for a total filtration area of 0.023 m². Vinasse was loaded in a feed tank, pumped circulating liquid at 5 m/s, with a trans-membrane pressure set at 400 kPa (4 bar). System was operated in batch. Permeate was extracted continuously until no more permeate flow is measured. Volumetric Concentration Factor was monitored [VCF=volume of feed/volume of retentate]. Permeate was collected to feed reverse osmosis.

Three membranes were tested, with cut-off sizes of 0.1 μm, 0.2 μm, and 0.45 μm. The membrane with cut-off size of 0.1 μm had highest flow rate and the least tendency to become plugged. Microfiltration was performed with membrane of cut-off size 0.1 μm until VCF reached 40. At the outset, flow rate was 175 l/hm²; at the conclusion, flow rate was 40 l/hm². Results were as follows:

| | volume | | conductivity | absorbance |
|---|---|---|---|---|
| | (l) | brix | (mS/cm) | at 420 nm |
| Feed | 20 | 4.8 | 12.7 | 18.9 |
| Permeate | 19.5 | 4.5 | 12.4 | 6.5 |
| retentate | 0.5 | 12.4 | 8.0 | 141.8 |

### EXAMPLE 2

### Reverse Osmosis (RO) (Step b))

Feed was the permeate from Example 1. The pilot plant was equipped with a 4000 kPa (40 bar), 1250 liter/hour piston pump, an RO/NF spiral housing module. Pressure was set with a backpressure needle valve, flowrate was controlled with a flowmeter. The feed was loaded in the feed tank, then concentrated until 4000 kPa (40 bar) was reached. System was operated in batch mode. Pressure was adjusted to maintain permeate flow below 100 liter/hour, to prevent bursting the element. VCF was registered until maximum operating pressure was reached. Operation was at constant pressure of 3000 kPa (30 bar). Membrane was a FILMTEC™ BW30-2540 membrane from Filmtec Corporation.

Flowrate reduced continuously with concentration increase, maximum VCF reached was 3.9. Average flowrate for this concentration was about 10 l/h.m². Membrane was just rinsed with water after the concentration test, flowrate performance was recovered without cleaning. Reverse osmosis test was performed on 19.5 liters, and reverse osmosis test duration was about 40 minutes.

After reverse osmosis, the retentate from the reverse osmosis process was subjected to evaporation to reduce the amount of water by approximately half. The results of microfiltration, reverse osmosis, and evaporation were as follows:

| | | MF | RO | RO | Concentrated |
|---|---|---|---|---|---|
| | Crude | Permeate | permeate | Retentate | retentate by |
| | vinasse | average | average | average | evaporation |
| Volume | 20 | 19.5 | 14.5 | 5.0 | 2.8 |
| liters | | | | | |
| Brix | 4.8 | 4.5 | 0.1 | 14.2 | 29.0 |
| pH | 4.8 | 4.8 | 5.2 | 4.8 | 4.8 |
| Color abs | 8.7 | 6.5 | 0.01 | 25.92 | 33.0 |
| 420 nm | | | | | |
| Turbidity abs | 11.2 | 0 | 0 | 0 | 0 |
| 420 nm | | | | | |
| Conductivity | 12.7 | 12.4 | 0.66 | 30.2 | 55.6 |
| mS/cm | | | | | |
| Salts g/l | 37 | 37 | 0.45 | 148.0 | 302.0 |
| Unknown | 5.7 | 5.7 | 0 | 22.0 | 45.0 |
| Organics g/l | | | | | |
| Glycerol g/l | 4.4 | 4.3 | 0 | 17.1 | 35.0 |
| Inositol g/l | 1.0 | 1.0 | 0 | 4.0 | 8.2 |

MF = microfiltration
RO = reverse osmosis

Brix was measured by refractometer by Belligham & Stanley at 20° C.
Turbidity was measured by by spectrophotometer at 420 nm wavelength using ICUMSA method GS 7-21 (2007), published by International Commission for Uniform Methods of Sugar Analysis (http://www.icumsa.org).
Conductivity was measured by _conductimeter by Hanna at 20° C.
Unknown organics was measured by HPLC using Biorad™ HPX 87K column and Water+ 0.13 g/l K₂HPO₄ as eluent at 0.6 ml/min, 70° C.
Glycerol was measured by HPLC using Biorad™ HPX 87C column and Water at 0.6 ml/min, 80° C.
Inositol was measured by HPLC using Biorad™ HPX 87C column and Water at 0.6 ml/min, 80° C.

### EXAMPLE 3

### Ion Exclusion Chromatography (Step c))

The chromatography column was 25*1000 mm glass with adjustable piston and jacket for temperature control, distribution with 25 μm PTFE frit. Total resin capacity was about 460 ml. A circulation water bath was used at 60° C., along with a peristaltic pump, and an autosampler.

The resins were Dowex™ 99320 resin and Amberlite™ CR1310 resin (both from the Dow Chemical Co.).

Resin Filling

The loading of resin was done in a column half filled with degassed demineralized water. The resin level was adjusted after heating till the appropriate temperature by recycling hot water during at least 30 min (flow=4 BV/h).

The resin was compacted before doing any separation by performing two pulse tests but without any sampling and data recording (flow=4 BV/h). The compaction is due to the swelling and the shrinking of the resin, following injection of product then water. After these two elutions, the resin level was adjusted to the top of the column.

The appropriate amount of product was loaded on the top of column, then it was displaced through the resin bed by water elution. The fractions were collected at the bottom of the column with a constant interval of volume (each 0.04 BV from 0.3 BV to 1.2 BV; depending on product affinity). The 0.3 first BV were sent to the drain. They were only water. At the outlet of the column, 20 samples were recovered and analyzed.

20 ml of Blue dextran (from Fluka) at 1 g/l were injected to measure the hydrodynamic efficiency of the column at the same flow rate as feed pulse test. Color was measured @625 nm. Blue dextran, glucose and fructose were injected at two different flowrates, 10 and 40 ml/min, to measure the effect of dispersion due to flow increase. 20 ml of Glucose and fructose were injected pure at 20% brix. The concentration of fractions are measured by brix determination.

The simple elution gives us the plot of each component (concentration versus effluent volume-BV), and these data were translated into separation coefficients. The starting point of the elution is defined as the middle of the feed injection, in order to reduce the effect of the load dispersion. The calculation formulas were as follows:

BV=Σ cᵢ*bvᵢ*d(bv)/Σ cᵢ*d(bv)

K=(BV−ε)/(1−ε)

σ²=[Σ cᵢ*bvᵢ²*d(bv)/Σ cᵢ*d(bv)]−BV²

H=L*σ²/(BV²)

R_{A/B}=2(BV_{B}−BV_{A})/(4[(σ_{A})+(σ_{B})])

where
ε=porosity of the resin bed, ratio of the interstitial volume between resin beads versus bed volume.
K=affinity coefficient of the product for the resin.
H=theorical plate height: it is a dispersion coefficient of the product for the resin. (cm)
bv=eluted volume per unit of column volume
BV=average retention volume for the product expressed per unit of column volume.
L=length of the column. (cm)
σ²=variance of the peak.
c=concentration
i=number of sample
d(bv)=sampling interval
R=Resolution

It is considered that ε is close to 0.36. Possibly, ε could be measured precisely by using a molecule which has no affinity for the resin, such as blue dextran. The average retention volume of blue dextran BV_{bd} is the porosity.

The eluent was measured for pH, conductivity, and absorbance at 420 nm Each fraction was also analyzed for salt content, for carbohydrate content (including glycerol and inositol), and for DP2 and organic acid content. DP2 is the amount of non-fermentable sugars.

In Test 1, the resin was Na+ form of Amberlite™ CR1310. Results were as follows. The salt peak began at 0.4 BV and ended at 0.65 BV. The peak of the eluent containing glycerol and inositol began at around 0.65 BV. There was almost no overlap between the two peaks. Analysis of these results showed the following:

| | salts & | DP2 & | glycerol & | |
|---|---|---|---|---|
| | color | organic acids | inositol | remainder |
| BVi | 0.529 | 0.715 | 0.808 | 0.786 |
| Ki | 0.214 | 0.525 | 0.680 | 0.644 |
| Hi cm | 1.648 | 1.307 | 1.045 | 3.862 |

Test 2 was a repeat of Test 1. Qualitatively, the peaks appeared the same as in Test 1. Analysis of the data from Test 2 showed the following:

| | salts & | DP2 & | glycerol & | |
|---|---|---|---|---|
| | color | organic acids | inositol | remainder |
| BVi | 0.5447 | 0.719 | 0.793 | 0.889 |
| Ki | 0.241 | 0.532 | 0.655 | 0.815 |
| Hi cm | 1.755 | 1.032 | 0.81 | 0.887 |

In both Test 1 and Test 2, DP2 and organic acids elute in between salts and glycerol+inositol peak. These components will be recovered partly with salts, partly with glycerol and inositol peak.

Test 3 used Dowex™ 99/320 resin in Na+ form.

Salts peak started earlier than with Amberlite™ CR1310 Na, but Inositol and Glycerol Peak also started earlier than with Amberlite™ CR1310 Na. Overlap is not larger than with CR1310 Na. Advantage of this Dowex™ 99/320 resin is glycerol peaks ends at 0.8 BV while we measured end at 0.95 BV with CR1310, so less elution volume is required. Analysis of the data showed the following:

| | salts & | DP2 & | glycerol & | |
|---|---|---|---|---|
| | color | organic acids | inositol | remainder |
| BVi | 0.4672 | 0.602 | 0.665 | 0.630 |
| Ki | 0.112 | 0.337 | 0.442 | 0.383 |
| Hi cm | 1.608 | 1.812 | 1.12 | 4.333 |

Test 4 was a repeat of Test 3, and the appearance of the peaks was the same. Analysis of Test 4 showed the following:

| | salts & | DP2 & | glycerol & | |
|---|---|---|---|---|
| | color | organic acids | inositol | remainder |
| BVi | 0.4672 | 0.602 | 0.665 | 0.630 |
| Ki | 0.112 | 0.337 | 0.442 | 0.383 |
| Hi cm | 1.608 | 1.812 | 1.12 | 4.333 |

Overlap between the salts peak and the glycerol+inositol peak is low.

To compare resins, we calculate resolution factors, as follows:

| | RESOLUTION TABLE | | | RESOLUTION TABLE | | |
|---|---|---|---|---|---|---|
| | Amberlite ™ CR1310 | | | DOWEX ™ 99/320 | | |
| | 8 < | 11.5 < | | 8 < | 11.5 < | |
| | RT < | RT < | | RT < | RT < | |
| | 10 | 11.8 | RT >12 | 10 | 11.8 | RT >12 |
| RT <8 | −0.597 | −0.873 | −0.818 | −0.465 | −0.760 | −0.489 |
| 8 < RT < 10 | 0.000 | −0.007 | −0.011 | 0.000 | −0.005 | −0.006 |
| 11.5 < RT < | 0.000 | 0.000 | −0.127 | 0.000 | 0.000 | 0.039 |
| 11.8 | | | | | | |

Resolution appears to be much better with CR1310, thanks to its higher humidity. We selected this resin for the separation. For the next step only CR1310 was tested.

Feed sample were collected on all four previous tests, high purity pools were mixed together. Pool sampling was as follows:

| | CR1310/1 | CR1310/2 | 99/320/1 | 99/320/2 | Average |
|---|---|---|---|---|---|
| Sample starts | 0.753 | 0.711 | 0.669 | 0.626 | |
| Sample ends | 0.966 | 0.902 | 0.796 | 0.775 | |
| Volume ml | 100 | 90 | 60 | 70 | |
| Brix | 0.63 | 0.57 | 0.64 | 0.45 | 0.57 |
| pH | 7.1 | 6.7 | 6.4 | 7.1 | 6.8 |
| Conductivity | 0.1 | 0.07 | 0.08 | 0.06 | 0.08 |
| color | 0.01 | 0.01 | 0.01 | 0.01 | 0.01 |
| DP2 % | | | | | 24.8 |
| Inositol % | | | | | 11.2 |
| Glycerol % | | | | | 48.2 |
| Unknown % | | | | | 16 |

From the 4 tests, 320 ml of product was pooled, with an average DS of 6 g/l containing approximately 60% of glycerol and inositol. DS is the amount of dry solids. The pool sample was concentrated up to 3% DS by evaporation before injection into Amberlite™ CR1310 Ca⁺⁺ resin for affinity chromatography. The pool sample was then treated with mixed bed of ion exchange resins for complete demineralization.

1. A process for extracting polycosanol from cane vinasse comprising
providing a cane vinasse concentrate, wherein the amount of organic compounds in said cane vinasse concentrate is 35 g/l or more, and wherein said cane vinasse concentrate is effectively free of solid particles having diameter of 10 μm or higher,
performing an ion exclusion chromatography step c) on said cane vinasse concentrate to produce an extract (EC) and a raffinate (RC),
performing a concentration step y) on said raffinate (RC) to produce a permeate (PY) and a retentate (RY), and
performing a separation step d)ii) on said retentate (RY) to produce a retentate (RDII) that contains polycosanol.

2. The process of claim 1, further comprising the step, after said concentration step y) and prior to said separation step d)ii), adding one or more organic solvent to said retentate (RY).

3. The process of claim 1, wherein said separation step d)ii) is performed by nanofiltration.

4. The process of claim 3, wherein said separation step d)ii) is conducted at pressure of 500 kPa to 8,000 kPa.

4. The process of claim 3, wherein said separation step d)ii) is conducted at temperature of 30° C. to 80° C.

5. The process of claim 1, wherein said step d)i) of performing affinity chromatography is performed, and wherein said process of claim 1 further comprises the step of removing glycerol from said extract (EDI).

6. The process of claim 1, wherein said cane vinasse concentrate is produced by a process comprising
a) filtering cane vinasse to produce a retentate (RA) and a permeate (PA),
b) concentrating said permeate (PA) to produce a permeate (PB) and a retentate (RB), wherein said retentate (RB) is said cane vinasse concentrate.
